(19) United States
(12) Patent Application Publication
(10) Pub. No.: US 2009/0133695 A1
(43) Pub. Date: May 28, 2009

(54) Mechanical ventilator system

(76) Inventors: CHAMKURKISHTIAH P. RAO, Mohawk, NY (US); DIANA C. LISTER, New Hartford, NY (US)

Correspondence Address: LITMAN LAW OFFICES, LTD., POST OFFICE BOX 15035, CRYSTAL CITY STATION, ARLINGTON, VA, 22215-0035, US

(21) Appl. No.: 12/076,751

(22) Filed: Mar. 21, 2008

Related U.S. Application Data
(60) Provisional application No. 60/996,615, filed on Nov. 27, 2007.

(51) Int. Cl. A61M 16/00; A61M 16/10

(52) U.S. Cl. 128/204.22; 128/204.23

(57) ABSTRACT

The mechanical ventilator system is a compact and portable artificial respiration system. A negative pressure vortex generator delivers an FiO₂ mix from an air-oxygen blender to the patient during the patient's inhalations, but remains idle during the patient's exhalations. Exhaust gases generated by the patient are released through an exhaust gas valve. During operation, the patient's oxygen saturation level is measured by an infrared pulse-oxygen probe, and an FiO₂ autoregulator is in communication with the probe to receive oxygen saturation level signals. The FiO₂ autoregulator is coupled with the air-oxygen blender to control the oxygen proportion of the FiO₂ mix. An automatic pressure flow sensor is fluidly coupled with the patient's airway to control actuation of the negative pressure vortex generator. The automatic flow sensor is coupled with a controller, which actuates a vortex generator trigger circuit in communication with the vortex generator.

### CROSS-REFERENCE TO RELATED APPLICATION

This application claims the benefit of U.S. Provisional Patent Application Ser. No. 60/996,615, filed Nov. 27, 2007.

### BACKGROUND OF THE INVENTION

1. Field of the Invention

The present invention relates to medical devices for respiratory therapy and treatment, and particularly to a mechanical ventilator system that provides a compact, portable ventilator for mechanically assisted respiration.

2. Description of the Related Art

In medicine, mechanical ventilation is a method of mechanically assisting or replacing autonomic breathing when patients cannot do so by themselves adequately. Mechanical ventilation typically follows invasive intubation with an endotracheal or tracheostomy tube, through which air is directly delivered to the patient's lungs. Typically, mechanical ventilation is used in acute settings such as in the Intensive Care Unit (ICU) for a short period of time during a serious illness. Conventional mechanical ventilation systems typically deliver gases into the patient's lungs with a pressure greater than the ambient atmospheric pressure. This is in contrast to older negative pressure ventilators, such as an “iron lung”, which generate a negative pressure environment around the patient's thorax to entrain gases into the patient's lungs. Iron lung ventilators are no longer used for typical mechanical ventilation.

Modern mechanical ventilators may be classified as pressure cycled, volume cycled, and high frequency oscillator types. These systems all develop some form of positive pressure to deliver the gases into the patient's lungs. The drawbacks of all of the above ventilators are: the use of positive pressures, which may lead to barotrauma to the lung tissue which leads to chronic lung disease (CLD); and inadequate regulation of inspired air/oxygen mixture (FiO₂). Low FiO₂ may cause hypoxemia, and high FiO₂ may cause direct oxygen toxicity to the lungs and remote toxicity to the eyes of the premature infants, which leads to Retinopathy of Prematurely (ROP), which may cause blindness and other eye lesions. These complications of present day ventilators are well known and demonstrated in the medical literature, particularly in the management and care of premature infants.

Further, although often a life saving technique, mechanical ventilation carries many potential complications including pneumothorax, airway injury, alveolar damage, and ventilator-associated pneumonia, among others. Thus, patients are typically weaned off mechanical ventilation as soon as possible.

Many different types of mechanical ventilators are presently in use. Examples of such ventiltors include transport ventilators, intensive care unit (ICU) ventilators, neonatal intensive care unit (NICU) ventilators (which are designed with the preterm neonate in mind; these are a specialized subset of ICU ventilators that are designed to deliver the smaller, more precise volumes and pressures required to ventilate these patients), and positive airway pressure (PAP) ventilators, which are specifically designed for non-invasive ventilation.

Because a mechanical ventilator is responsible for assisting in a patient's breathing, it must be able to deliver an adequate amount of oxygen in each breath. The “fraction of inspired oxygen” (FiO₂) represents the percent of oxygen in each breath that is inspired. Normal room air has approximately 21% oxygen content by volume. In adult patients who can tolerate higher levels of oxygen for a period of time, the initial FiO₂ may be set at 100% until arterial blood gases can document adequate oxygenation. An FiO₂ of 100% for an extended period of time can be dangerous, but it can protect against hypoxemia from unexpected intubation problems. For infants, and especially in premature infants, avoiding high levels of FiO₂ (>60%) is important.

Positive end-expiratory pressure (PEEP) is an adjunct to the mode of ventilation used in cases where the functional residual capacity (FRC) is reduced. At the end of expiration, the PEEP exerts pressure to oppose passive emptying of the lung and to keep the airway pressure above the atmospheric pressure. The presence of PEEP opens up collapsed or unstable alveoli and increases the FRC and surface area for gas exchange, thus reducing the size of the shunt. Thus, if a large shunt is found to exist based on the estimation from 100% FiO₂, then PEEP can be considered and the FiO₂ can be lowered (<60%) to still maintain an adequate PaO₂, thus reducing the risk of oxygen toxicity.

In addition to treating a shunt, PEEP is also therapeutic in decreasing the work of breathing. In pulmonary physiology, compliance is a measure of the “stiffness” of the lung and chest wall. The mathematical formula for compliance (C)=change in volume/change in pressure. Therefore, a higher compliance means that only small increases in pressure can lead to large increases in volume, which means the work of breathing, is reduced. As the FRC increases with PEEP, the compliance also increases, since the partially inflated lung takes less energy to inflate further.

In neonatal patients, CLD and ROP are of great concern. As noted above, NICU mechanical ventilators are typically positive pressure mechanical ventilators, converted for use with neonatal infants. CLD and ROP may be caused by barotrauma (which may be caused by positive pressure ventilators) and hyperoxia. A negative pressure ventilator with auto-regulation of FiO₂ would aid in avoiding barotrauma, hypoxemia and hyperoxemia. Further, conventional mechanical ventilators, as described above, are typically bulky, often consisting of various pieces of equipment which take up an entire room's worth of space. Such a system is not easily transportable, particularly in emergency situations. Thus, a mechanical ventilator system solving the aforementioned problems is desired.

### SUMMARY OF THE INVENTION

The mechanical ventilator system includes a negative pressure vortex generator in fluid communication with an air oxygen blender for delivering oxygen to a patient. The system is preferably portable and provides a controllable oxygen flow to a patient, ranging from neonatal patients to adults. The system is actuated by the inspiratory effort of the patient. The inspiratory effort of the patient generates a negative air pressure in the range of approximately 4 mm to 6 mm Hg or greater. During the expiratory phase, the mechanical ventilator remains idle, allowing the patient to exhale exhalation gases via an exhalation valve (as will be described in greater detail below) with minimal resistance.

A suitable sensor or measuring device, such as an infrared pulse-oxygen probe, is used for measuring oxygen saturation in a patient's blood. The sensor is in communication with a controller that regulates the fraction of inspired oxygen (FiO₂) of the output oxygen from the air-oxygen blender. The controller is preferably a pre-set processor or other control in communication with the sensor through wires, cables, a wireless electromagnetic interface or the like. The controller is preferably a real-time FiO₂ autoregulator. The real-time FiO₂ autoregulator communicates directly with the air-oxygen blender through wires, cables, a wireless electromagnetic interface or the like.

The air-oxygen blender receives air from the environment or compressed air, and oxygen from a pure oxygen source and outputs the FiO₂ mix. The FiO₂ mix is delivered to the patient by the negative pressure vortex generator. A pressure flow gauge may be positioned along the flow path, allowing the user to manually control the pressure of the FiO₂ mix being delivered to the patient.

An automatic flow sensor, which may be pre-set to detect pressure or carbon dioxide levels in the FiO₂ mix being delivered to the patient, is preferably positioned further along the flow path. The automatic flow sensor is in communication with a vortex generator control (which may be a programmable logic controller or the like), which drives a vortex generator trigger circuit to operate the negative pressure vortex generator. Further, the inspiratory effort of the patient also triggers the automatic flow sensor, which, in turn, generates a triggering signal for the actuation of the negative pressure vortex generator (through the vortex generator control and the vortex generator trigger circuit).

As noted above, exhalations from the patient pass through an expiratory valve, allowing for the release of exhaust gasses from the patient. Further, a mechanism for controlling positive end-expiratory pressure of expired air from the patient is provided, and is preferably coupled to the expiratory valve. The PEEP control mechanism may be a control knob or the like, which is attached to a valve coupled with the expiratory valve.

In an alternative embodiment, the conventional air-oxygen blender is coupled with a stepper motor (either through an external mechanical coupling, or with the air-oxygen blender and the stepper motor being an integral unit). In this embodiment, the real-time FiO₂ autoregulator includes two separate controllers, namely, a pulse-oxygen controller and a separate stepper motor controller, with each being in communication with the other. The two separate controllers may be formed as an integral control unit, which is further in communication with a display (such as a liquid crystal display or the like), allowing the patient's heart rate, oxygen saturation or any other desired information to be displayed to the user. The display is coupled to the integral control unit through wires, cables, a wireless interface or the like.

The stepper motor controller is in communication with the stepper motor (through wires, cables, a wireless interface or the like), and the controlled FiO₂ mix is delivered to the patient from the air-oxygen blender by any suitable delivery mechanism, such as the negative pressure vortex generator, as described above.

These and other features of the present invention will become readily apparent upon further review of the following specification and drawings.

### BRIEF DESCRIPTION OF THE DRAWINGS

FIG. 1 is a block diagram of a mechanical ventilator system according to the present invention.

FIG. 2 is a block diagram of an alternative embodiment of the mechanical ventilator system according to the present invention.

Similar reference characters denote corresponding features consistently throughout the attached drawings.

### DETAILED DESCRIPTION OF THE PREFERRED EMBODIMENTS

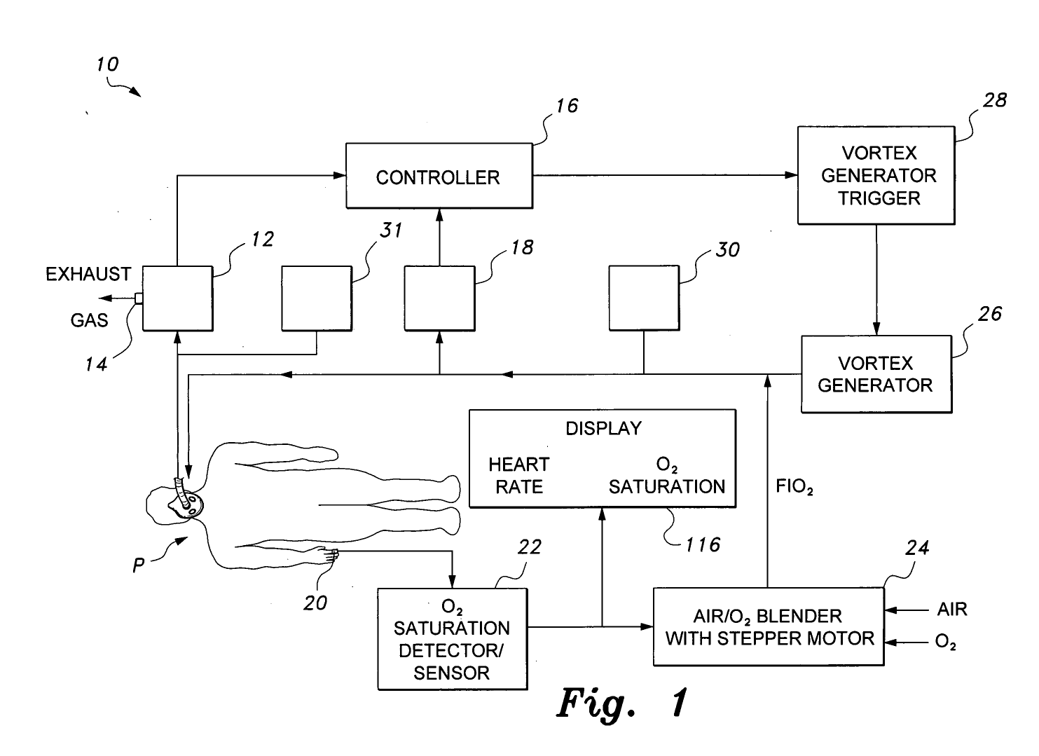

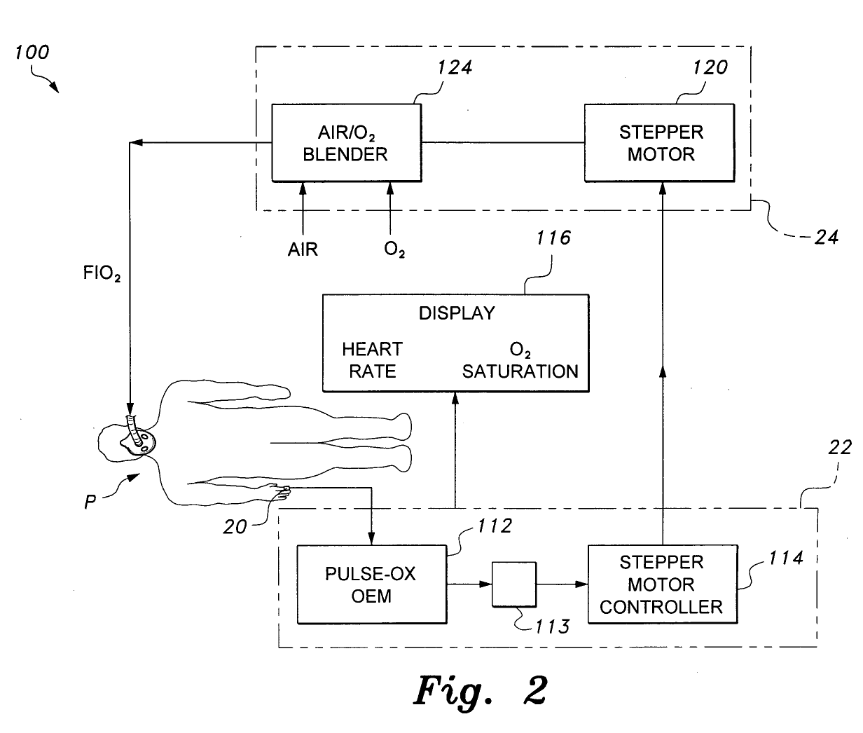

The present invention is directed towards a mechanical ventilator system 10. As best shown in FIGS. 1 and 2, the mechanical ventilator system 10 includes a negative pressure vortex generator 26 in fluid communication with an air oxygen blender 24 for delivering oxygen to a patient. The system is preferably portable and provides a controllable oxygen flow to a patient, ranging from neonatal patients to adults. The system is actuated by the inspiratory effort of the patient. The inspiratory effort of the patient generates a negative air pressure in the range of approximately 4 mm to 6 mm Hg or greater. During the expiratory phase, the mechanical ventilator 10 remains idle, allowing the patient to exhale exhalation gases via an exhalation valve 14 (as will be described in greater detail below) with minimal resistance. Preferably, vortex generator 26, auto-regulated air/oxygen blender 24, the timing control mechanism (controller) 16, and the digital display 116 are all encased within a portable housing for compactness and portability of the ventilator system 10. This system may be adapted for use for patient age ranges from premature infants to adults.

Air-oxygen blenders are well known in the art, and air-oxygen blender 24 may be any conventional air-oxygen blender. Examples of conventional air-oxygen blenders are shown in U.S. Pat. Nos. 3,727,827; 3,895,642; and 5,014,694, the disclosures of which are hereby incorporated by reference.

A suitable sensor or measuring device, such as an infrared pulse-oxygen probe 20 is used for measuring oxygen saturation in a patient's blood. The sensor is in communication with a controller that regulates the fraction of inspired oxygen (FiO₂) of the output oxygen from the air-oxygen blender. The controller is preferably a pre-set processor or other control in communication with the sensor through wires, cables, a wireless electromagnetic interface or the like. The controller is preferably a real-time FiO₂ autoregulator 22. The real-time FiO₂ autoregulator 22 communicates directly with the air-oxygen blender 24 through wires, cables, a wireless electromagnetic interface or the like. Depending upon the measured oxygen-saturation level in patient P, measured by sensor 20, the FiO₂ autoregulator 22 generates control signals, which are received by air-oxygen blender 24 to produce an FiO₂ mix having the desired and necessary proportion of oxygen, depending upon pre-set parameters.

The real-time autoregulation of blended oxygen is achieved through the use of an oxygen saturation measuring device, such as a pulse-oxygen sensor, which is well-known in the art. Preferably, a miniaturized pulse-oxygen sensor is incorporated in the microprocessor controlled stepper motor driver unit 22, to be described below. The data received from the oxygen saturation sensor is processed by the microcontroller and sends instructions to the stepper motor driver which, in turn, drives the stepper motor in the desired direction to obtain desired mixture of oxygen/air in the inspired gases to keep the patient's oxygen saturation in the normal range.

The air-oxygen blender 24 receives air from the environment and oxygen from a pure oxygen source (such as bottled, pressurized oxygen, for example) and outputs the FiO₂ mix, as indicated by the directional arrow in FIG. 1. The FiO₂ mix is delivered to the patient P by the negative pressure vortex generator 26, along a flow path which feeds directly to the patient P. A pressure flow gauge 30 may be positioned along the flow path, allowing the user to manually measure and control the pressure of the FiO₂ mix being delivered to the patient. Pressure flow gauge 30 may be any conventional gas pressure flow gauge.

An automatic flow sensor 18, which may be pre-set to detect pressure or carbon dioxide levels in the FiO₂ mix being delivered to patient P, is preferably positioned further along the flow path, as shown. Automatic flow sensor 18 may be any suitable, conventional pressure or carbon dioxide sensor. The automatic flow sensor is in communication with a vortex generator control 16 (which may be a programmable logic controller or the like), which drives a vortex generator trigger circuit 28 to operate the negative pressure vortex generator 26. Further, the inspiratory effort of the patient P also triggers the automatic flow sensor 18, which, in turn, generates a triggering signal for the actuation of the negative pressure vortex generator 28 (through the vortex generator control 16 and the vortex generator trigger circuit 28). Automatic flow sensor 18 can measure changes in pressure generated by the inhalations of the patient, thus triggering delivery of the FiO₂ mix.

As noted above, the vortex generator system consists of a vortex generator 26, controller 16 and at least one sensor 18, along with pressure relief valves 14 and exhalation valves 12, positioned within the gas delivery circuit. The sensor or sensors 18 are placed at the proximal end of the gas delivery circuit, preferably near the ET tube, nose or face mask. These sensors 18 may be used to measure the pressure, flow or carbon dioxide in the expired gases, and this data is then fed into the controller 16. The data may be used to display the pressure in the gas delivery circuit, and also as trigger input data for the controller 16 to trigger the vortex generator trigger 28, which controls the vortex generator 26. The vortex generator 26 is only triggered during the inspiratory phase, during which the patient generates the required negative pressure, and the vortex generator 26 augments the delivery of the gases to the patient's alveoli. This delivery facilitates better gas exchange in the alveoli.

As noted above, exhalations from the patient P pass through an expiratory valve 14, allowing for the release of exhaust gasses from the patient. Expiratory valve 14 may be any suitable, conventional exhaust valve. Further, a mechanism for controlling positive end-expiratory pressure (PEEP) of expired air from the patient 12 is provided, and is preferably coupled to the expiratory valve 14, as shown. The PEEP control mechanism 12 may be a control knob or the like, which is attached to a valve coupled with the expiratory valve 14.

The vortex generator 26 maintains the negative pressure throughout the inspiratory phase, which simulates normal breathing, thereby avoiding barotrauma to the lung tissue. The respiratory cycle is essentially under the patient's control, and the vortex ventilator system augments the patient's efforts in the inspiratory phase. The vortex generator 26 can be powered by AC or DC electricity, additional electromechanical means, such as solenoids, pneumatic drivers, oscillators, piston pumps, electric or pneumatic reciprocating device, or linear actuators acoustic speakers with square wave generators. As will be described in greater detail below, an LCD display 116 is used to show heart rate and oxygen saturation. Similar LCD displays may be used to show FiO₂ levels, the inspiratory and expiratory pressures and respiratory rate, and other relevant data.

In an alternative embodiment 100, illustrated in FIG. 2, the conventional air-oxygen blender 24 is coupled with a stepper motor 120, either through an external mechanical coupling, or with the air-oxygen blender 24 and the stepper motor 120 being formed as an integral unit 118. In the embodiment of FIG. 2, the real-time FiO₂ autoregulator (which replaces regulator 22 of FIG. 1) includes two separate controllers, namely, a pulse-oxygen OEM (a standard component, which is a conventional system in mechanical ventilators) 112 and a separate stepper motor controller 114, with each being in communication with the other. The two separate controllers 112, 114 may be formed as an integral control unit (as shown by the dashed-line box in FIG. 2), which is further in communication with a display 116 (such as a liquid crystal display or the like), allowing the patient's heart rate, oxygen saturation or any other desired information to be displayed to the user. The display 116 is coupled to the integral control unit 110 through wires, cables, a wireless interface or the like.

The stepper motor controller 114 is in communication with the stepper motor 120 (through wires, cables, a wireless interface or the like), and the controlled FiO₂ mix is delivered to the patient from the air-oxygen blender 24 by any suitable delivery means, such as the negative pressure vortex generator, described above. The control means 112, 114 may be programmable logic controllers or any other suitable processors or control device.

In system 100, the stepper motor 120 controls the oxygen proportionality module of the air-oxygen blender 24. In use, the infrared pulse-oxygen sensor 20, positioned on the patient, measures the oxygen saturation of the blood of the patient P, and communicates this measured level to the pulse-oxygen OEM 112. This, in turn, drives the stepper motor controller 114 to drive stepper motor 120. Preferably, the system 100 is formed as a compact, portable unit.

In use, and with particular regard to the embodiment of FIG. 2, the ventilator system ventilates the patient's lungs during the inspiratory phase (i.e., the negative pressure phase of the respiratory cycle). The ventilator then idles during expiratory phase. If the negative pressure ventilation is inadequate to maintain proper gas exchange, the system can be used as a positive pressure ventilator by controlling the exhalation valves 14. When the patient's lung function improves, the patient may be weaned to negative pressure ventilation in order to minimize the possibility of barotrauma to the lungs. During both the positive and the negative pressure ventilation, the inspired oxygen (FiO₂) is regulated via a closed loop to maintain adequate oxygenation, thereby minimizing the oxygen toxicity and hypoxemia.

The infrared pulse-oxygen sensor 20 is typically applied to patient's digit or ear lobe in order to detect the patient's pulse rate and the level of oxygen saturation. The signal from pulse-oxygen sensor 20 is conveyed to the FiO₂ regulator 22.

The FiO₂ regulator 22 preferably includes a built-in pulse-oxygen saturation software controller system 112 coupled with a pulse-oxygen data processor 113. The pulse-ox OEM 112 and the pulse-oxygen data processor 113 form an integral pulse-ox controller system, coupled with controller 114. The digital data of the oxygen saturation level and heart rate generated by system 112 is processed by the pulse-oxygen processor 113. The output from the pulse-oxygen processor 113 is used to drive the stepper motor controller 114, which commands the stepper motor 120. The stepper motor regulates the Air/O₂ blender 124 output to deliver the required inspired oxygen (FiO₂) to the patient in order to maintain the desired oxygenation in the patient's blood. This is accomplished in real time with minimal lag time. Preferably, the system regulates the FiO₂ with each heart beat. It should be understood that additional safety features may be added to the FiO₂ regulator 100 in order to safeguard against any possible malfunctions or failure.

A flow/pressure or carbon dioxide sensor 18 is located proximally to the patient in the inspiratory path of the gas delivery/exhaust circuit. The signal form the sensor 18 is communicated to the controller 16. The controller 16 then triggers the vortex generator 26, via the vortex generator-trigger 28, during the inspiratory phase of the respiratory cycle. The vortex generator 26 remains idle during the expiratory phase. Thus, the vortex generator cycling is governed by the patient's respiratory effort and assists in the delivery of FiO₂ during the inspiratory phase.

The FiO₂ output from the air/O₂ blender with stepper motor 24 is fed into the inspiratory path of the gas delivery/exhaust circuit. There is a minimal continuous flow of FiO₂ during the idle phase of the vortex generator 26. One or more pressure gauges are located close to the patient in the inspiratory part 30 and the expiratory part 31 of the gas delivery/exhaust circuit. This allows medical personnel to monitor the pressures generated during the inspiratory phase of ventilator operation.

Safety valves 14 are placed in the gas delivery circuit in order to relieve any unexpected rise in pressure, and there are further valves included in the circuit that are used if positive pressure modality is preferred.

It is to be understood that the present invention is not limited to the embodiments described above, but encompasses any and all embodiments within the scope of the following claims.

We claim:

1. A mechanical ventilator system, comprising:
a negative pressure vortex generator;
means for measuring oxygen saturation in a patient's blood;
an air-oxygen blender for outputting oxygen to the patient;
means for delivering the output from the air-oxygen blender to the patient, the means for delivering the output being in fluid communication with the negative pressure vortex generator and the air-oxygen blender, the means for delivering the output from the air-oxygen blender to the patient being actuated during the patient's inhalations and being idle during the patient's exhalations;
means for controlling a fraction of inspired oxygen in the output from the air-oxygen blender, the means for controlling being in communication with the means for measuring oxygen saturation in a patient's blood; and
means for controlling positive end-expiratory pressure of expired air from the patient, the means for controlling positive end-expiratory pressure being in communication with the negative pressure vortex generator.

2. The mechanical ventilator system as recited in claim 1, wherein the means for controlling the fraction of inspired oxygen in the output from the air-oxygen blender comprises a stepper motor.

3. The mechanical ventilator system as recited in claim 1, wherein the means for measuring oxygen saturation in the patient's blood comprises a sensor adapted to be worn by the patient.

4. The mechanical ventilator system as recited in claim 3, wherein the sensor comprises an infrared pulse oxygen probe.

5. The mechanical ventilator system as recited in claim 1, further comprising means for selectively actuating said negative pressure vortex generator.

6. The mechanical ventilator system as recited in claim 5, further comprising an automatic flow sensor in communication with the means for selectively actuating said negative pressure vortex generator.

7. The mechanical ventilator system as recited in claim 6, wherein the automatic flow sensor is a pressure sensor.

8. The mechanical ventilator system as recited in claim 6, wherein the automatic flow sensor is a carbon dioxide sensor.

9. The mechanical ventilator system as recited in claim 1, further comprising means for measuring and controlling the output from the air-oxygen blender being delivered to the patient.

10. The mechanical ventilator system as recited in claim 9, wherein the means for measuring and controlling the output comprises at least one pressure gauge.

11. The mechanical ventilator system as recited in claim 1, further comprising a display in communication with the air-oxygen blender and the means for measuring oxygen saturation in a patient's blood.

12. A mechanical ventilator system, comprising:
means for measuring oxygen saturation in a patient's blood;
an air-oxygen blender for outputting oxygen to the patient;
means for delivering the output from the air-oxygen blender to the patient, the means for delivering the output being in fluid communication with the air-oxygen blender, the means for delivering the output being actuated during the patient's inhalations and being idle during the patient's exhalations;
a stepper motor coupled with the air-oxygen blender for controlling a fraction of inspired oxygen in the output from the air-oxygen blender, the stepper motor being in communication with the means for measuring oxygen saturation in a patient's blood; and
means for displaying the oxygen saturation of the patient's blood.

13. The mechanical ventilator system as recited in claim 12, further comprising a negative pressure vortex generator, the negative pressure vortex generator being in fluid communication with the means for delivering the output from the air-oxygen blender to the patient.

14. The mechanical ventilator system as recited in claim 12, wherein the means for measuring oxygen saturation in a patient's blood comprises a sensor adapted to be worn by the patient.

15. The mechanical ventilator system as recited in claim 14, wherein the sensor is an infrared pulse oxygen probe.

16. The mechanical ventilator system as recited in claim 15, further comprising a pulse oxygen controller in communication with the infrared pulse oxygen probe and the stepper motor.

17. The mechanical ventilator system as recited in claim 16, further comprising a stepper motor controller in communication with the pulse oxygen controller and the stepper motor.

18. The mechanical ventilator system as recited in claim 17, further comprising means for processing pulse-oxygen data in communication with the pulse oxygen controller and the stepper motor controller, the means for processing pulse-oxygen data transmitting control signals to the stepper motor controller responsive to measured levels of the oxygen saturation of the patient's blood.

19. The mechanical ventilator system as recited in claim 12, further comprising means for measuring the patient's heart rate.

20. The mechanical ventilator system as recited in claim 19, further comprising means for displaying the patient's heart rate.
